(19) United States
(12) Patent Application Publication
(10) Pub. No.: US 2018/0272090 A1
(43) Pub. Date: Sep. 27, 2018

(54) SYSTEM AND METHOD FOR PROTECTING A PATIENT'S TRACHEAL WALL DURING PERCUTANEOUS PROCEDURES

(71) Applicant: Hansa Medical Products, Inc., Carmel, IN (US)

(72) Inventor: Eric D. Blom, Carmel, IN (US)

(21) Appl. No.: 15/470,229

(22) Filed: Mar. 27, 2017

(51) Int. Cl. A61M 16/04

(52) U.S. Cl. CPC A61M 16/0465; A61M 16/04; A61M 16/0434

(57) ABSTRACT

A medical instrument system and method including a tracheo-protective shield that extends outwardly from an endotracheal tube positioned in a patient's trachea is disclosed.

### TECHNICAL FIELD

The present disclosure relates generally to medical instruments and methods, and in particular to a medical instrument and method for a percutaneous procedure.

### BACKGROUND

A tracheal tube is typically a catheter that is inserted into the trachea for the purpose of providing an airway for a patient. An endotracheal tube is a specific type of tracheal tube that is inserted through the mouth (orotracheal) or nose (nasotracheal). Such endotracheal tubes are in contrast to a tracheostomy tube, which is inserted into a tracheostoma following a tracheostomy procedure, and a tracheal button, which may also be inserted into a puncture through the paratracheal skin into the trachea.

An endotracheal tube may be cuffed or uncuffed. Cuffed endotracheal tubes include a cuff that can be inflated to minimize the passage of secretions from the upper respiratory tract downward into the lungs of a patient.

The following are of interest: U.S. Pat. Nos. 1,598,283; 2,892,458; 3,688,774; 3,996,939; 4,211,234; 4,223,411; 4,280,492; 4,304,228; 4,305,392; 4,315,505; 4,327,721; 4,449,523; 4,459,984; 4,469,100; 4,573,460; 4,584,998; 4,589,410; 4,596,248; 4,607,635; 4,627,433; 4,632,108; 4,637,389; 4,762,125; 4,834,087; 4,840,173; 4,852,565; 5,056,515; 5,067,497; 5,107,828; 5,123,922; 5,201,310; 5,217,008; 5,218,970; 5,255,676; 5,297,546; 5,329,921; 5,339,808; 5,343,857; 5,349,950; 5,391,205; 5,392,775; 5,458,139; 5,497,768; 5,507,279; 5,515,844; 5,584,288; 5,599,333; RE35,595; 5,687,767; 5,688,256; 5,746,199; 5,771,888; 5,957,978; 6,053,167; 6,089,225; 6,102,038; 6,105,577; 6,135,110; 6,135,111; 6,463,927; 6,722,367; 6,814,007; 7,404,329; 8,573,220; U.S. Patent Publication Nos: 2003/0084905; 2004/0123868; 2016/028239; 2016/0102312; foreign/international patent publications: DE 25 05 123; DE 34 06 294; DE 37 20 482; DE 38 13 705; DE 195 13 831; DE 101 09 935; WO 99/07428; WO 99/12599; WO 00/32262; other publications: Quick Reference Guide to Shiley's “Quality-Of Life” Line of Tracheostomy Products, 1991; Granuloma Associated with Fenestrated Tracheostomy Tubes, Padmanabhan Siddharth, MD, PhD, FACS and Lawrence Mazzarella, Md., FACS, Case Reports, vol. 150, August 1985, pp. 279-280; Technical Support Information Connections with the Passy-Muir Tracheostomy and Ventilator Speaking Valves, one sheet; Tracheostomy and Laryngectomy Tubes, pp. 568 and 572; Tracheostomy Tube Adult Home Care Guide, Shiley Tracheostomy Products, Mallinckrodt Medical pp. 1-40; D. Hessler, MD, K. Rehder, MD and S. W. Karveth, MD, “Tracheostomy Cannula for Speaking During Artificial Respiration”, Anesthesiology, vol. 25, No. 5, pp. 719-721 (1964). No representation is intended by this listing that a thorough search of all material prior art has been conducted, or that no better art than that listed is available.

### SUMMARY

According to one aspect of the disclosure, a medical instrument system is disclosed. The medical instrument system comprises a tissue shield sized to be advanced into a lumen of an endotracheal tube. The tissue shield includes a barrier plate sized and shaped to extend outwardly the distal end of the endotracheal tube and block a medical tool from contacting a posterior wall of the patient's trachea. The system may also comprise an endotracheal tube including a distal end sized to be positioned in a patient's trachea, a proximal end configured to extend outwardly from one of the patient's mouth and the patient's nose, and a lumen extending between the distal end and the proximal end. In some embodiments, the medical tool may be a cutting tool such as, for example, a percutaneous needle. Additionally, in some embodiments, the barrier plate to sized and shaped to block the medical tool from puncturing a posterior wall of the patient's trachea.

In some embodiments, the system may also comprise an elongated tube sized to be positioned in the lumen of the endotracheal tube. The tissue shield may include a cylindrical body sized to be received a passageway defined in the elongated tube. The barrier plate may be secured to, and extend distally away from, the cylindrical body.

In some embodiments, the elongated tube may include a plurality of corrugations, and the cylindrical body of the tissue shield may include a rib sized to be positioned in one of the corrugations to secure the shield to the elongated tube. Additionally, in some embodiments, system may comprise a medical instrument including a cable sized to be advanced into the lumen of the endotracheal tube. The tissue shield may be secured to a distal end of the cable of the surgical instrument.

In some embodiments, the medical instrument includes a laryngoscope. In some embodiments, the cable may be a stylette.

Additionally, in some embodiments, the system may comprise an elongated tube sized to be positioned in the lumen of the endotracheal tube. The tissue shield may be secured to a distal end of the elongated tube. In some embodiments, the elongated tube and the tissue shield may be formed as a single monolithic component. In some embodiments, the tissue shield may be configured to be detached from the elongated tube.

In some embodiments, the barrier plate may include a curved wall that extends around a longitudinal axis from a first edge to a second edge, and an opening is defined between the first edge and the second edge of the curved wall, the opening being sized cover a predetermined portion of a patient's soft tissue. Additionally, in some embodiments, the curved wall may be formed from a semi-flexible plastic. In some embodiments, the curved wall may be formed from silicone.

In some embodiments, the endotracheal tube may include an inflatable cuff positioned between the distal end and the proximal end.

According to another aspect, a medical instrument system comprises an elongated tube sized to be positioned in an endotracheal tube, the elongated tube having a distal end, a proximal end, and an outer surface devoid of any openings between the distal end and the proximal end. The system also comprises a tissue shield secured to the distal end of the elongated tube. The shield includes a barrier plate sized and shaped to block a cutting tool from puncturing a posterior wall of the patient's trachea.

According to another aspect, a medical instrument system comprises an elongated tube sized to be positioned in an endotracheal tube, the elongated tube having a distal end, a proximal end, and an outer surface devoid of any openings between the distal end and the proximal end. The system also comprises a tissue shield secured to the distal end of the elongated tube. The shield includes a barrier plate sized and shaped to block a medical tool from contacting a posterior wall of the patient's trachea.

In some embodiments, the elongated tube and the tissue shield may be formed as a single monolithic component. Additionally, in some embodiments, the tissue shield may be configured to be detached from the elongated tube.

In some embodiments, the tissue shield may include a cylindrical body sized to be received a passageway defined in the elongated tube, and the barrier plate may be secured to, and extend distally away from, the cylindrical body.

In some embodiments, the elongated tube may include a plurality of corrugations, and the cylindrical body of the tissue shield may include a rib sized to be positioned in one of the corrugations to secure the shield to the elongated tube.

According to another aspect, a medical instrument system comprises a surgical instrument including a cable sized to be advanced into an endotracheal tube, and a tissue shield secured to a distal end of the cable. The tissue shield includes a barrier plate sized and shaped to prevent a cutting tool from puncturing a posterior wall of the patient's trachea.

According to another aspect, a method of forming an incision in a patient's neck in advance of implanting a tracheal device is disclosed. The method comprises sliding an endotracheal tube to move the distal end of the endotracheal tube proximally within the patient's trachea, moving a tissue shield outward from the distal end of the endotracheal tube to position a barrier plate of the shield in the patient's trachea between an anterior wall of the patient's trachea and a posterior wall of the patient's trachea, inserting a medical or surgical tool through the anterior wall of the patient's trachea to form an incision, and advancing the surgical tool into engagement with the barrier plate of the shield.

In some embodiments, the method also comprises withdrawing an elongated tube from the endotracheal tube, and attaching the tissue shield to a distal end of the elongated tube. The step of moving the shield outward from the distal end of the endotracheal tube may include inserting the elongated tube with the tissue shield attached to its distal end into the endotracheal tube.

In some embodiments, the method may comprise inserting the tissue shield attached to a distal end of a laryngoscope into the endotracheal tube, and advancing the tissue shield and the distal end of the laryngoscope toward the distal end of the endotracheal tube.

In some embodiments, the method may further comprise inserting the tissue shield attached to a distal end of a stylette into the endotracheal tube, and advancing the tissue shield and the distal end of the stylette toward the distal end of the endotracheal tube.

According to another aspect, a method of forming an incision in a patient's neck in advance of implanting a tracheal device is disclosed. The method comprises positioning a tissue shield in a patient's trachea between an anterior wall of the patient's trachea and a posterior wall of the patient's trachea, inserting a medical or surgical tool through the anterior wall of the patient's trachea to form an incision, and advancing the surgical tool into engagement with the tissue shield.

In some embodiments, positioning the tissue shield in the patient's trachea between the anterior wall of the patient's trachea and the posterior wall of the patient's trachea may include advancing the tissue shield along the outer surface of another medical instrument. Additionally, in some embodiments, the medical instrument may be an endotracheal tube. In some embodiments, the method may comprise inserting the tissue shield attached to a distal end of a laryngoscope into the patient's trachea. In some embodiments, the method may further comprise inserting the tissue shield attached to a distal end of a stylette into the patient's trachea.

In some embodiments, the method also comprises withdrawing an elongated tube from an endotracheal tube, and attaching the tissue shield to a distal end of the elongated tube. The step of positioning the tissue shield in the patient's trachea between the anterior wall of the patient's trachea and the posterior wall of the patient's trachea may include inserting the elongated tube with the tissue shield attached to its distal end into the endotracheal tube.

In some embodiments, the method may comprise inserting the tissue shield attached to a distal end of a laryngoscope into the endotracheal tube, and advancing the tissue shield and the distal end of the laryngoscope toward the distal end of the endotracheal tube.

In some embodiments, the method may further comprise inserting the tissue shield attached to a distal end of a stylette into the endotracheal tube, and advancing the tissue shield and the distal end of the stylette toward the distal end of the endotracheal tube.

### BRIEF DESCRIPTION OF THE DRAWINGS

The detailed description particularly refers to the following figures, in which:

FIG. 1 is an exploded perspective view of a system for use in a percutaneous procedure;

FIG. 2 is a perspective view of a tissue shield of the system of FIG. 1;

FIG. 3 is an anterior elevation view of the tissue shield of FIG. 2;

FIG. 4 is an anterior elevation view of the system of FIG. 1;

FIG. 5 is a perspective view of the system of FIG. 4;

FIGS. 6-12 are cross-sectional side elevation views showing the system of FIGS. 1-5 positioned in a patient's body during a percutaneous tracheostomy;

FIG. 13 is a perspective view of another embodiment of a tissue shield and an elongated tube for use with the system;

FIG. 14 is a perspective view of additional embodiments of a tissue shield;

FIG. 15 is a perspective view showing the tissue shield and the elongated tube of FIG. 13 being positioned for insertion into the endotracheal tube of FIG. 1;

FIG. 16 is a perspective view of a medical instrument with a tissue shield; and

FIG. 17 is a perspective view of another medical instrument with a tissue shield.

### DETAILED DESCRIPTION OF THE DRAWINGS

While the concepts of the present disclosure are susceptible to various modifications and alternative forms, specific exemplary embodiments thereof have been shown by way of example in the drawings and will herein be described in detail. It should be understood, however, that there is no intent to limit the concepts of the present disclosure to the particular forms disclosed, but on the contrary, the intention is to cover all modifications, equivalents, and alternatives falling within the spirit and scope of the invention as defined by the appended claims.

Terms representing anatomical references, such as anterior, posterior, medial, lateral, superior, inferior, etcetera, may be used throughout the specification in reference to the surgical tools and medical instruments described herein as well as in reference to the patient's natural anatomy. Such terms have well-understood meanings in both the study of anatomy and the field of orthopaedics. Use of such anatomical reference terms in the written description and claims is intended to be consistent with their well-understood meanings unless noted otherwise.

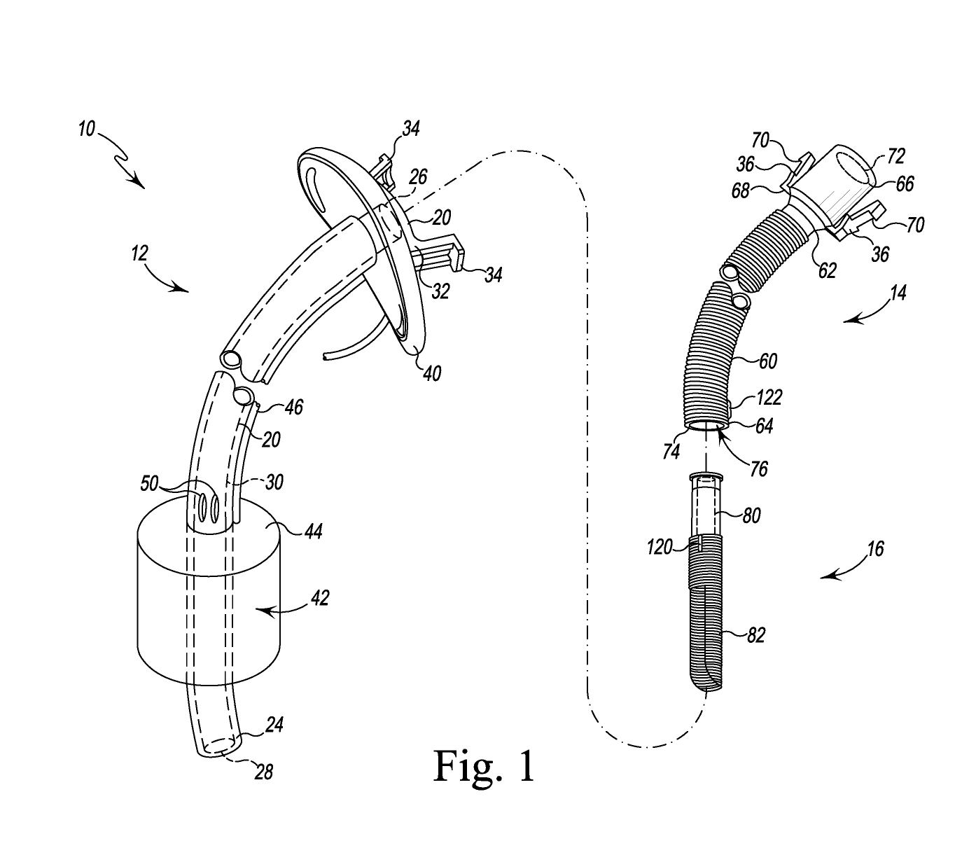

Referring now to FIG. 1, an system 10 is shown. In the illustrative embodiment, the system 10 is an orotracheal tube system configured for insertion through a patient's mouth into the patient's trachea. It should be appreciated that the structure and techniques described herein may be used with a nasotracheal tube as well. The system 10 includes an outer endotracheal tube 12, an inner elongated tube 14 configured to be inserted into the outer tube 12, and a tissue shield 16 configured to be attached to the inner elongated tube 14. As described in greater detail below, the system 10 may be used during a percutaneous procedure, such as, for example, a tracheostomy procedure, to prevent contact between the patient's posterior tracheal wall and a percutaneous needle, catheter, or other medical or surgical tool. In that way, the system 10 may be used in procedure for forming an incision in a patient's neck in advance of implanting a tracheal device. As used herein, a “tracheal device” includes tracheostomy tubes, sleep apnea devices, micro-tracheal devices, and other devices configured to be inserted into a patient's tissue through an tracheostoma or other incision in a patient's neck.

The outer endotracheal tube 12 includes an elongated curved body 20 that extends from a proximal end 22 to a distal end 24. The curved body 20 has a circular opening 26 defined in the proximal end 22 and another circular opening 28 defined in the opposite distal end 24. A lumen 30 extends between the openings 26, 28 and has a curvature corresponding to the curvature of the body 20. The tube 12 is rigid and formed from a biocompatible plastic resin, but it should be appreciated that in other embodiments other suitable materials may be used.

The outer endotracheal tube 12 has a rim 32 that extends outwardly from the proximal end 22. A pair of locking flanges 34 extend outwardly from the rim 32 on opposite sides of the proximal opening 26. As described in greater detail below, the flanges 34 are sized to receive corresponding locking tabs 36 of the elongated tube 14 to secure the elongated tube 14 to the endotracheal tube 12. In the illustrative embodiment, the rim 32, the locking flanges 34, and the elongated curved body 20 are formed as a single monolithic component. It should be appreciated that in other embodiments they may be formed as separate components that are later assembled.

As shown in FIG. 1, a guard plate 40 is attached to the elongated curved body 20 adjacent to the proximal end 22. In the illustrative embodiment, the guard plate 40 is attached via a pair of pivot pins (not shown) that permit limited movement of the guard plate 40 relative to the rest of the endotracheal tube 12. The guard plate 40 is oblong and sized to be positioned over a patient's mouth to keep the proximal end 22 of the outer endotracheal tube 12 outside of and offset from the patient's mouth when in use. The guard plate 40 is also formed from a biocompatible plastic resin but in the illustrative embodiment is semi-flexible.

In the illustrative embodiment, an inflatable cuff 42 is attached to the curved body 20 near the distal end 24 such that the cuff 42 lies in the patient's trachea during use. As shown in FIG. 1, the cuff 42 includes a sleeve 44 formed from silicone or other flexible material. The cuff 42 is inflatable through a conduit 46 that extends from the cuff 42 along the outer surface of the curved body 20 and beyond the proximal end 22. As described in greater detail below, the cuff 42 is inflatable through the conduit 46 when positioned in the patient's trachea to minimize the passage of secretions from the upper respiratory tract downward into the lungs of the patient. It should be appreciated that in other embodiments the inflatable cuff may be omitted.

As shown in FIG. 1, the outer endotracheal tube 12 includes a number of openings or fenestrations 50 that are positioned proximal of the inflatable cuff 42. Although two openings 50 are shown in FIG. 1, it should be understood that in other embodiments additional or fewer openings 50 may be included in the tube 12. In still other embodiments, the openings may be omitted.

As described above, the endotracheal tube system 10 also includes an inner elongated tube 14 configured for insertion into the outer tube 12. As shown in FIG. 1, the elongated tube 14 includes a corrugated body 60 that extends from a proximal end 62 to a distal end 64. The body 60 has a plurality of corrugations defined in its outer surface, which is devoid of any openings between the ends 62, 64 in the illustrative embodiment. The elongated tube 14 also includes a port 66 that extends proximally from the proximal end 62 of the corrugated body 60. A mounting flange 68 extends outwardly from the proximal end 62. A pair of locking tabs 36 extend outwardly and proximally from the mounting flange 68. Each tab 36 includes a raised edge 70 configured to engage a corresponding locking flange 34 of the outer endotracheal tube 12 to secure the elongated tube 14 in the outer tube 12.

The elongated tube 14 as a proximal opening 72 defined in the port 66 and an opposite distal opening 74 defined in the end 64 of the corrugated body 60. A passageway 76 extends between the openings 72, 74 and is sized to permit the passage of oxygen and other gases from a ventilator (not shown) or other medical device attached to the port 66 downward into the patient's lungs. In the illustrative embodiment, the corrugated body 60 and the port 66 are formed as a single semi-flexible monolithic component from a biocompatible plastic resin. In that way, the corrugated body 60 conforms to the shape of the curved lumen 30 of the outer tube 12 when the corrugated body 60 is positioned in the lumen 30. In the illustrative embodiment, the flange 68 and locking tabs 36 are rigid and formed separately from biocompatible plastic resin.

Although only a single elongated tube 14 is shown in FIG. 1, the system 10 includes multiple such tubes 14 so that the inner elongated tube may be replaced as needed and with less difficulty and without the trauma that might accompany the replacement of the outer endotracheal tube 12. It should be appreciated that the system 10 may include other elongated tubes of different configurations, which are sized to be positioned separately in the outer tube 12. For example, the system 10 may include an elongated tube having one or more openings positioned between its proximal and distal ends. The openings may be positioned such that secretions pooling at the fenestrations 50 of the outer tube 12 may be exposed to suction. Additionally, in other embodiments, the elongated tubes may not be corrugated and/ or semi-flexible.

As described above, the system 10 also includes a tissue shield 16 that is configured to be attached to the inner tube 14. The tissue shield is configured to be positioned in the patient's trachea to prevent contact between various medical instruments used in percutaneous procedures and the patient's posterior tracheal wall, as described in greater detail below. It should be appreciated that in other embodiments the tissue shield and the elongated tube may be formed as a single monolithic component rather than as separate components as shown in FIG. 1. In still other embodiments, the tissue shield may be configured to be attached to the endotracheal tube and may be configured to advance along the outer surface of the endotracheal tube into position in the patient's trachea.

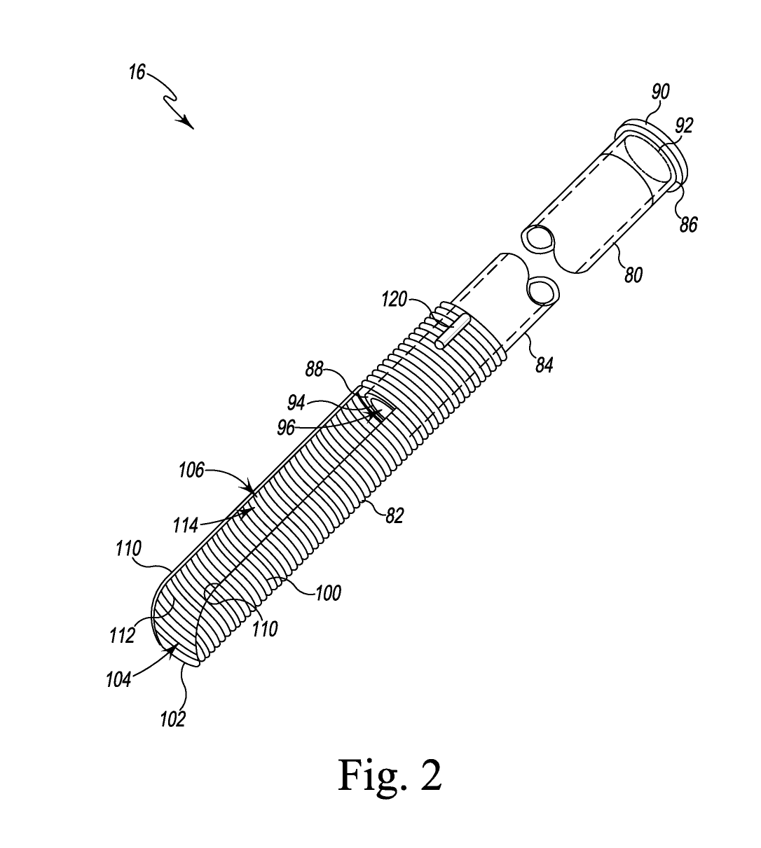

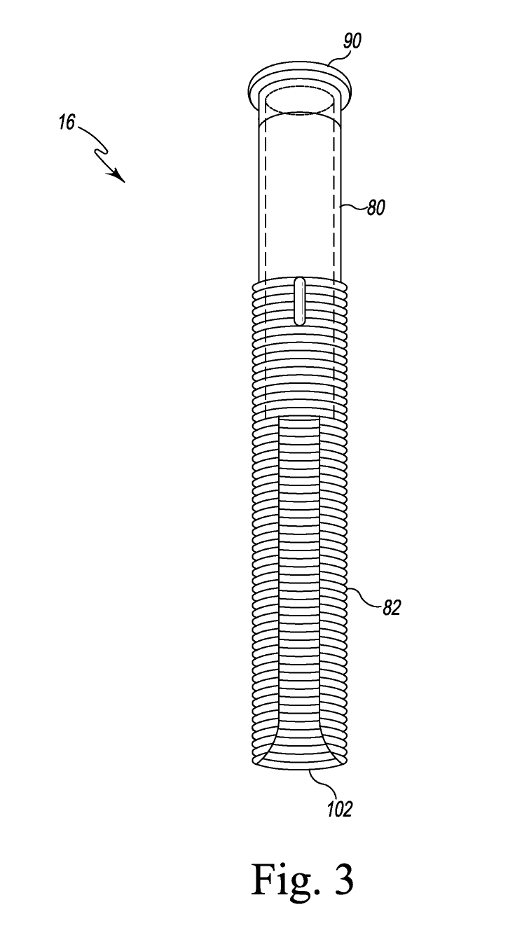

As shown in FIGS. 1-3, the tissue shield 16 is configured to be attached to the distal end 64 of the inner tube 14. In the illustrative embodiment, the tissue shield 16 includes a plug 80 and a barrier plate 82 that extends proximally from the plug 80. Referring now to FIG. 2, the plug 80 includes a cylindrical body 84 that extends from a proximal end 86 to a distal end 88. An annular rib 90 extends outwardly from the proximal end 86 and is configured to be received in one of the corrugations of the corrugated body 60 to attach the tissue shield 16 to the inner tube 14. The plug 80 also includes an opening 92 defined in the proximal end 86 and another opening 94 defined in its distal end 88. A passageway 96 extends between the openings 92, 94. As described in greater detail below, the passageway 96 is sized to permit the passage of an laryngoscope or other instrument such that movement of the percutaneous needle may be monitored from inside the patient's trachea.

The barrier plate 82 of the tissue shield 16 extends from the distal end 88 of the cylindrical body 84. In the illustrative embodiment, the barrier plate 82 includes a corrugated body 100 that extends to a distal tip 102. The corrugated body 100 has an open end 104 at the distal tip 102 and an elongated slot 106 that extends proximally from the open end 104. The slot 106 is defined between a pair of edges 110 of the barrier plate 82, and the barrier plate 82 has a curved inner wall 112 that connects the edges 110. As shown in FIG. 2, the inner wall 112 defines a channel 114 in the corrugated body 100 that opens into the passageway 96 of the plug 80. The elongated slot 106 is sized to receive the cutting tip of a percutaneous needle or other cutting tool, as described in greater detail below.

In the illustrative embodiment, the plug 80 is rigid and is formed from a bio compatible plastic resin. The barrier plate 82 is semi-flexible and is also formed from a biocompatible plastic resin. In the illustrative embodiment, the barrier plate 82 is formed from silicone. In other embodiments, it may be formed from any medical-grade material. The barrier plate 82 and the plug 80 are formed separately before being assembled as the tissue shield 16. It should be appreciated that in other embodiments the barrier plate and the plug may be formed as a single monolithic component.

As described above, the tissue shield 16 may be attached to the elongated tube 14 for insertion into the endotracheal tube 12. To do so, a surgeon or other user may align the proximal end 86 of the tissue shield 16 with the distal opening 74 of the elongated tube 14. The user may then advance the proximal end 86 into the distal opening 74 to engage the annular rib 90 of the tissue shield 16 with the corrugations of the elongated tube 14. The user may continue to advance the tissue shield 16 proximally until the proximal edge of the barrier plate 82 engages the distal end 64 of the elongated tube 14.

The tissue shield 16 includes an alignment guide 120 that is formed on the outer surface of the barrier plate 82, as shown in FIG. 2. In the illustrative embodiment, the alignment guide 120 is a single etch mark extending longitudinally along the outer surface of the barrier plate 82. The elongated tube 14 includes a corresponding alignment guide 122 on its outer surface. In illustrative embodiment, the user aligns the guides 120, 122 to orient the tissue shield 16 relative to the elongated tube 14 such that the elongated slot 106 faces anteriorly. It should be appreciated that in other embodiments the tissue shield, the elongated tube, and/or the endotracheal tube may include other guides to assist in the tissue shield is properly aligned for use in the surgical procedure.

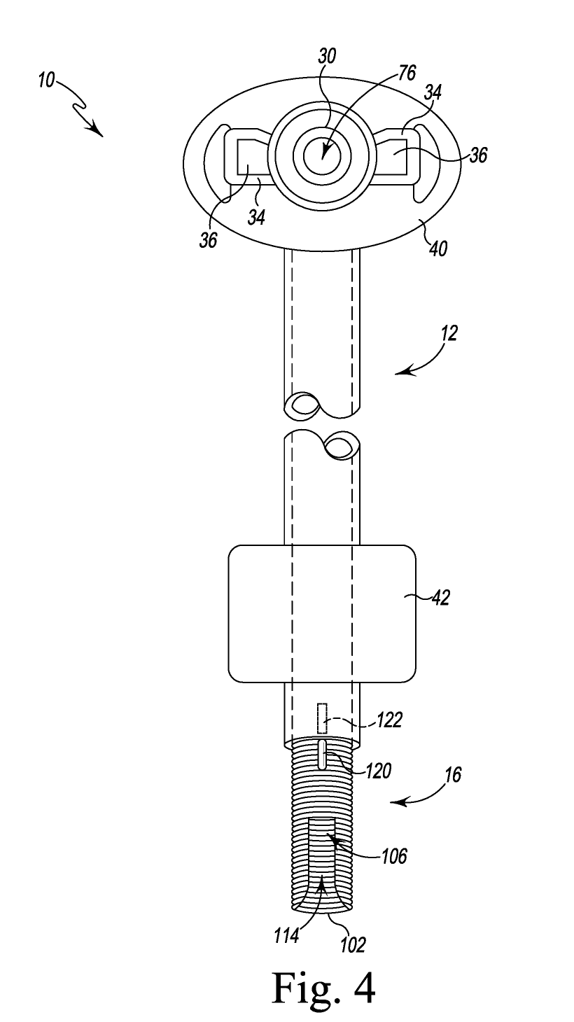

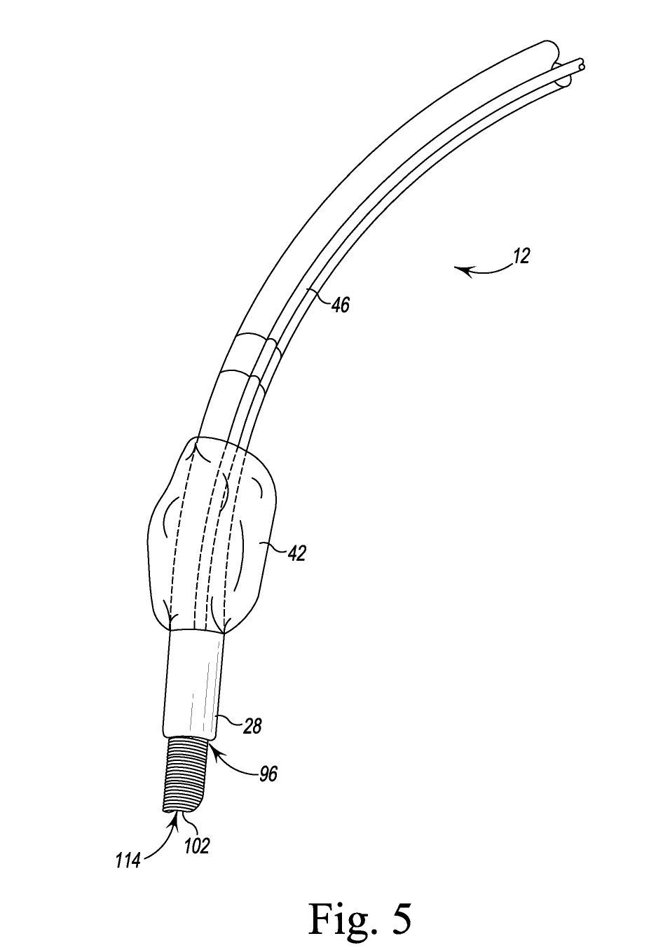

The user may then align the distal tip 102 of the tissue shield 16 with the proximal opening 26 of the endotracheal tube 12. The user may advance the distal tip 102 into the opening 26, along the lumen 30 of the endotracheal tube 12, and out of the distal opening 28 of the endotracheal tube 12. As shown in FIGS. 4-5, the elongated tube 14 and the tissue shield 16 are sized so that the tissue shield 16 extends outwardly from the distal opening 28 of the endotracheal tube 12 when the elongated tube 14 is secured to the tube 12 via the engagement of the locking tabs 36 with the flanges 34 of the tube 12. Because the tissue shield 16 is properly oriented relative to the elongated tube 14, the elongated slot 106 faces anteriorly so that it may receive the percutaneous needle or other cutting tool during the surgical procedure.

As described above, the system 10 includes multiple inner elongated tubes 14 for use with the outer endotracheal tube 12 so that the endotracheal tube 12 may remain positioned in a patient's trachea while the inner tube 14 is removed for cleaning and/or replacement with another inner tube.

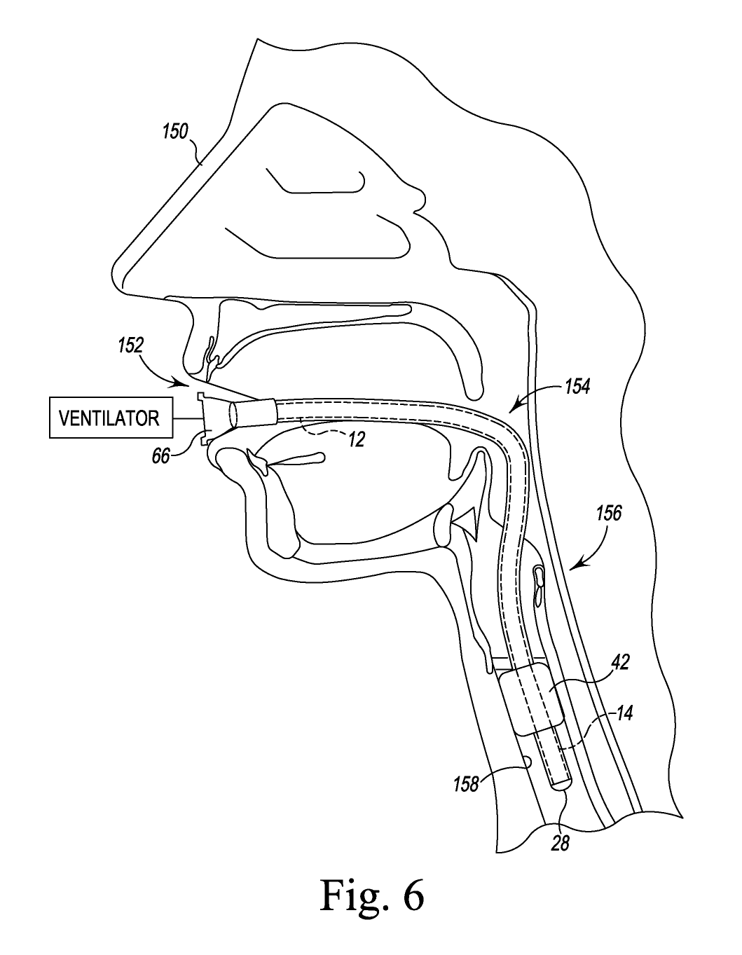

Referring now to FIG. 6, a patient 150 requiring a percutaneous tracheostomy is shown. An endotracheal tube 12 extends through the mouth 152 of the patient 150, down the patient's pharynx 154, and through the patient's glottis 156 so that the distal opening 28 of the tube 12 is positioned in the patient's trachea 158. As described above, the inflatable cuff 42 is attached to the curved body 20 of the endotracheal tube 12 and is positioned so that the cuff 42 lies in the trachea 158 below the glottis 156 when positioned as shown in FIG. 6.

An inner elongated tube 14 is shown positioned in the lumen 30 of the endotracheal tube 12. When positioned with its locking tabs 36 engaged with the flanges 34 of the endotracheal tube 12, the distal opening 74 of the elongated tube 14 is positioned adjacent to the distal opening 28 of the tube 12. As shown in FIG. 6, a ventilator or other device may be coupled to the port 66 of the inner elongated tube 14 to provide oxygen or other gases to the patient 150.

During the percutaneous tracheostomy, the inner elongated tube 14 may be removed from the outer endotracheal tube 12. The inner elongated tube 14 can be replaced by disconnecting it from the ventilator, releasing the locking tabs 136 from the flanges 134 and withdrawing the elongated tube 14 from the lumen 30 of the outer endotracheal tube 12. The surgeon or other user may then attach the tissue shield 16 to the distal end of the withdrawn elongated tube 14 as described above, or the surgeon may attach the tissue shield 16 to a new/ fresh elongated tube 14. The surgeon may then insert the tissue shield 16 and the elongated tube 14 into the lumen 30 of the outer endotracheal tube 12 as described above.

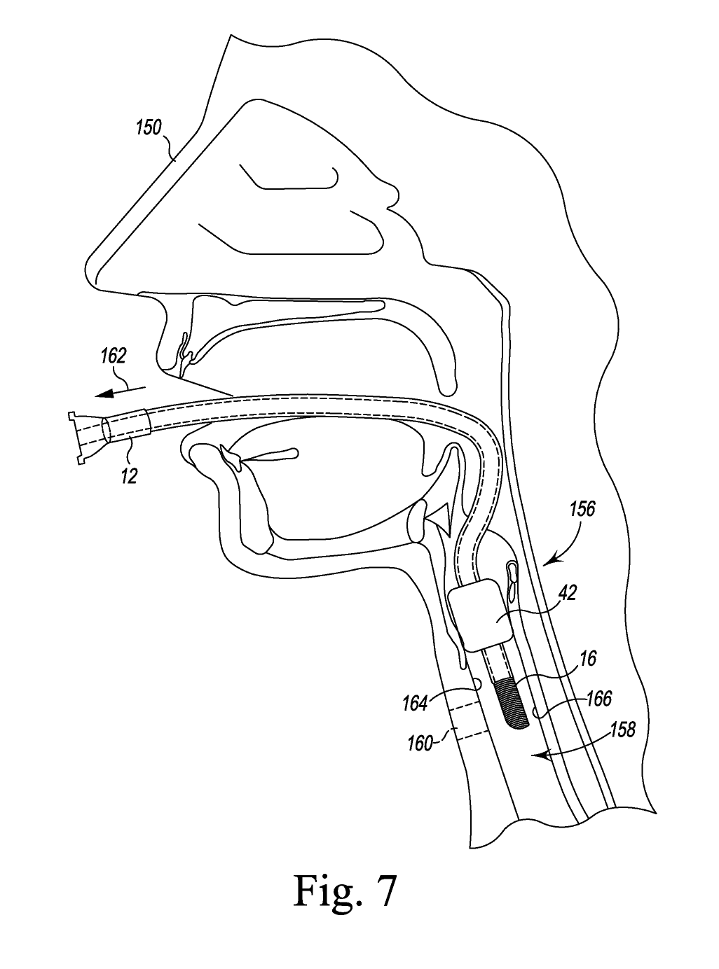

As shown in FIG. 7, the surgeon may also slide the endotracheal tube 12 proximally during the percutaneous tracheostomy procedure to position the distal end of the endotracheal tube 12 above the target area 160 for the tracheostomy incision. To do so, the surgeon may partially deflate the cuff 42 and then draw the endotracheal tube 12 proximally (as indicated by arrow 162) to move the cuff 42 into the patient's glottis 156. The surgeon may then re-inflate the cuff 42 to retain the endotracheal tube 12 in position above the target area 160. With the endotracheal tube 12 positioned as shown in FIG.7, the surgeon may advance the tissue shield 16 along the endotracheal tube 12 and outward from the distal opening 28 so that the barrier plate 82 is positioned in the trachea 158 between the target area 160 of the patient's anterior tracheal wall 164 and the patient's posterior tracheal wall 166.

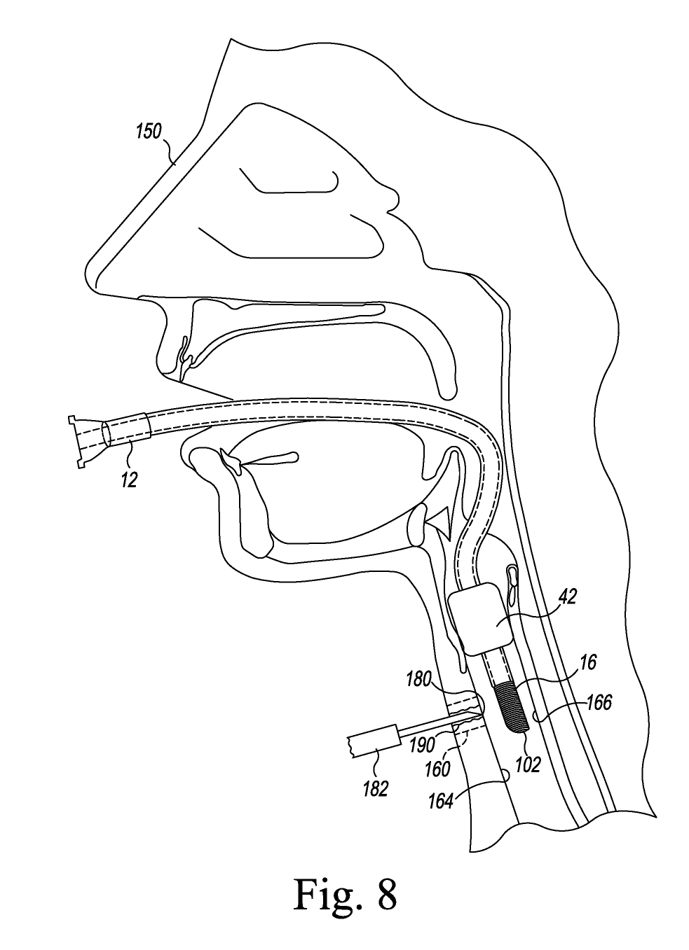

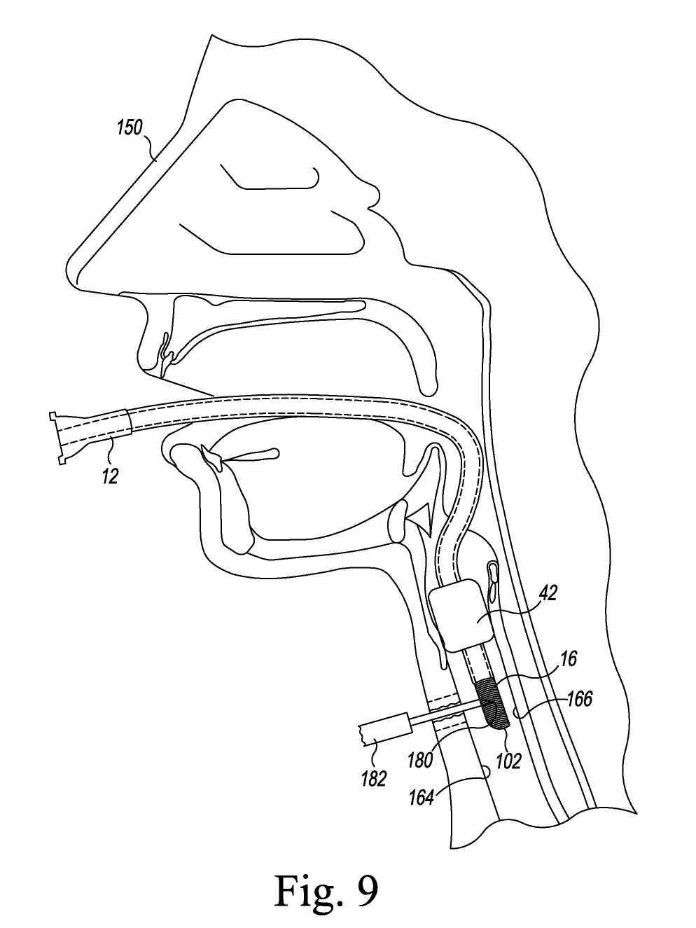

As shown in FIG. 8, the surgeon may then align the cutting tip 180 of a needle 182 with the target area 160 of the patient's neck and advance the cutting tip 180 into the tissue of the patient's neck. As shown in FIG. 9, the surgeon may advance the cutting tip 180 through the tissue in the patient's neck to form an incision 190 in the patient's anterior tracheal wall 164. As the cutting tip 180 is moved anterior to posterior within the patient's trachea 158, it is advanced into the elongated slot 106 of the tissue shield 16 and into the channel 114. If the cutting tip 180 is moved farther within the patient's trachea 158 toward the posterior tracheal wall 166, the tissue shield 16 covers and/ or redirects the cutting tip 180 to prevent or block contact between the cutting tip 180 and the posterior tracheal wall 166. In that way, the tissue shield 16 protects the posterior tracheal wall 166 and prevents inadvertent tissue damage.

The surgeon may then continue with the remaining steps of the percutaneous tracheostomy procedure. For example, the surgeon may advance a cannula 192 over the needle 182 and into the incision 190. The surgeon may then introduce a guidewire 194 through the cannula 192, as shown in FIG. 9. As the guidewire 194 advances through the patient's trachea 158, the tissue shield 16 is positioned to prevent the guidewire 194 from contacting the posterior tracheal wall 166. As shown in FIG. 9, the guidewire 194 may engage the tissue shield 16, which redirects the guidewire 194 away from the posterior tracheal wall 166 and down the trachea 158.

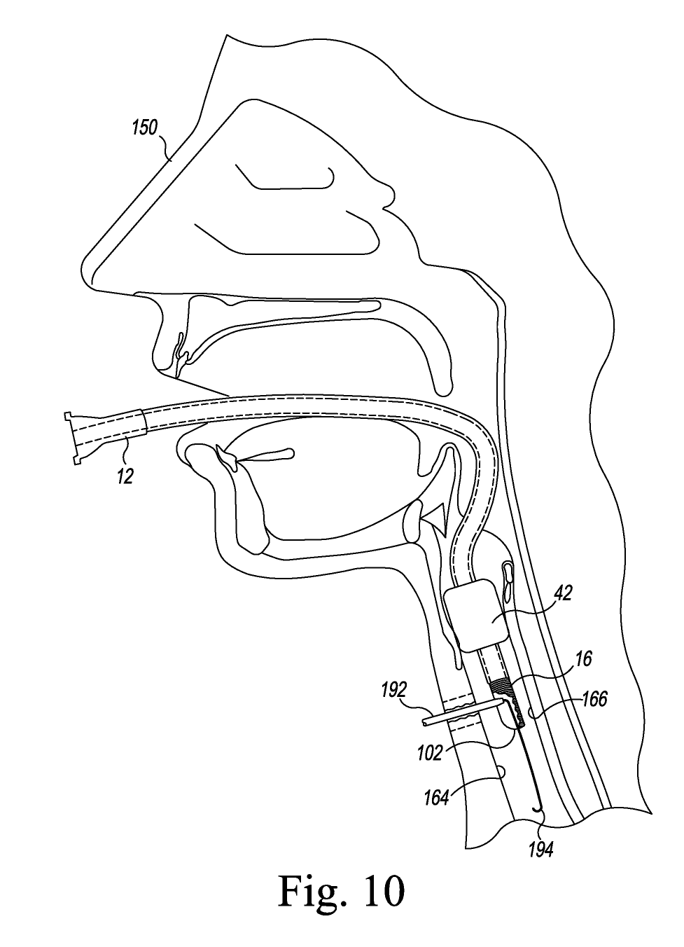

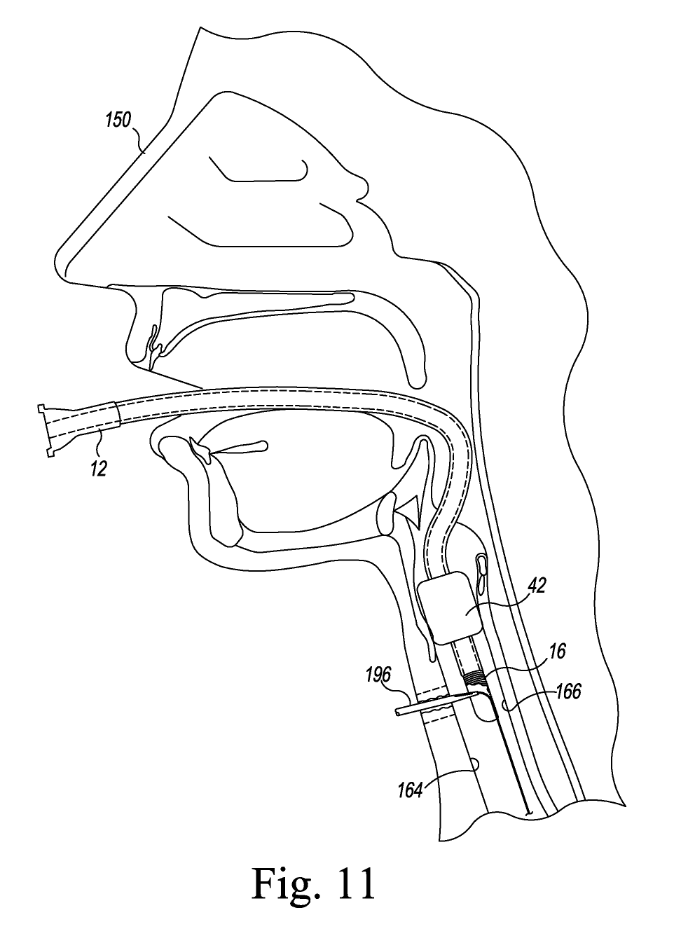

As shown in FIG. 10, the surgeon may also engage in pre-dilation of the incision 190 to expand the incision using a pre-dialator 196. Similar to the guidewire 194 and the needle 182, the tissue shield 16 is positioned in the trachea 158 to prevent contact with the posterior tracheal wall 166. As shown in FIG. 11, the surgeon may also insert a guiding catheter 198 into the expanded incision 190 with the tissue shield 16 positioned in the trachea 158 to prevent contact between the catheter 198 and the posterior tracheal wall 166. A tracheostomy tube may then be inserted into the expanded incision.

As described above, the passageways 76, 96 of the elongated tube 14 and the tissue shield 16, respectively, are sized to receive a probe of a laryngoscope. In that way, the surgeon may observe the movement of the needle 182 relative to the patient's trachea 158 through the laryngoscope. Additionally, surgeon may perform the other steps of the tracheostomy using the laryngoscope to observe the region of interest within the patient's trachea 158.

As described above, the tissue shield 16 may be formed as a single monolithic component with the elongated tube 14. As shown in FIG. 10, one embodiment of a tissue shield 216 includes a barrier plate 282 that is connected to a distal end 264 of an elongated tube 214. Similar to the barrier plate 82, the barrier plate 282 extends to a distal tip 284. The barrier plate 282 has a curved inner wall 286 that is connected to the distal end 264 of the elongated tube and defines a channel 288. The barrier plate 282 has an elongated slot 290 that is defined between the edges 292 of the curved inner wall 286. The slot 290 opens into the channel 288, as shown in FIG. 10.

In the illustrative embodiment, the barrier plate 282 is configured to contract such that it may be positioned in the lumen 30 of the endotracheal tube 12 (see FIG. 11). When the barrier plate 282 extends out of the distal opening of the lumen 30, as shown in FIG. 10, the barrier plate 282 expands or opens to fill an area below the endotracheal tube 12 that is greater in width than the width of the endotracheal tube 12. The slot 290 and channel 288 are sized to receive the cutting tip of a cutting tool such as, for example, a percutaneous needle.

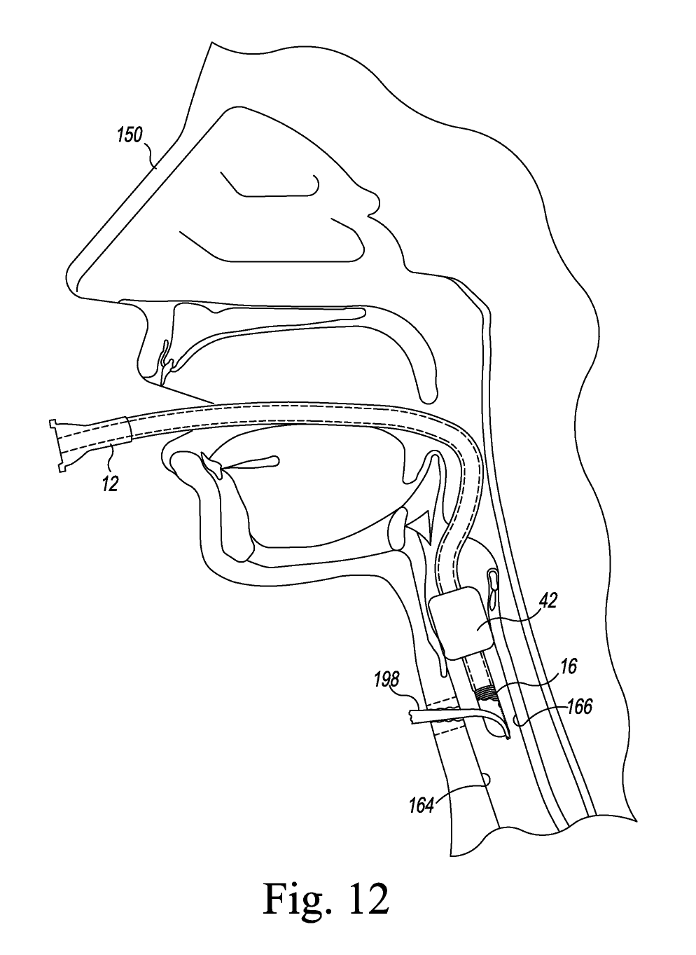

Referring now to FIG. 12, other embodiments of tissue shields 316, 416 are shown. The tissue shield 316 includes a solid plug 318 that may be attached to, for example, the probe of an endoscope or other medical instrument. The shield 316 also includes a barrier plate 320 that is formed rigid from a biocompatible resin. Similar to the barrier plates 82, 282, the barrier plate 320 extends to a distal tip 322. The barrier plate 320 has a curved inner wall 330 that is connected to the plug 318 and defines a channel 324. The barrier plate 320 has an elongated slot 326 that is defined between the edges 328 of the curved inner wall 330. The slot 326 and channel 324 are sized to receive the cutting tip of a cutting tool such as, for example, a percutaneous needle.

The tissue shield 416 also includes a solid plug 318 that may be attached to, for example, the probe of an endoscope or other surgical instrument. The shield 416 also includes a barrier plate 282 including the features described above in regard to FIG. 10.

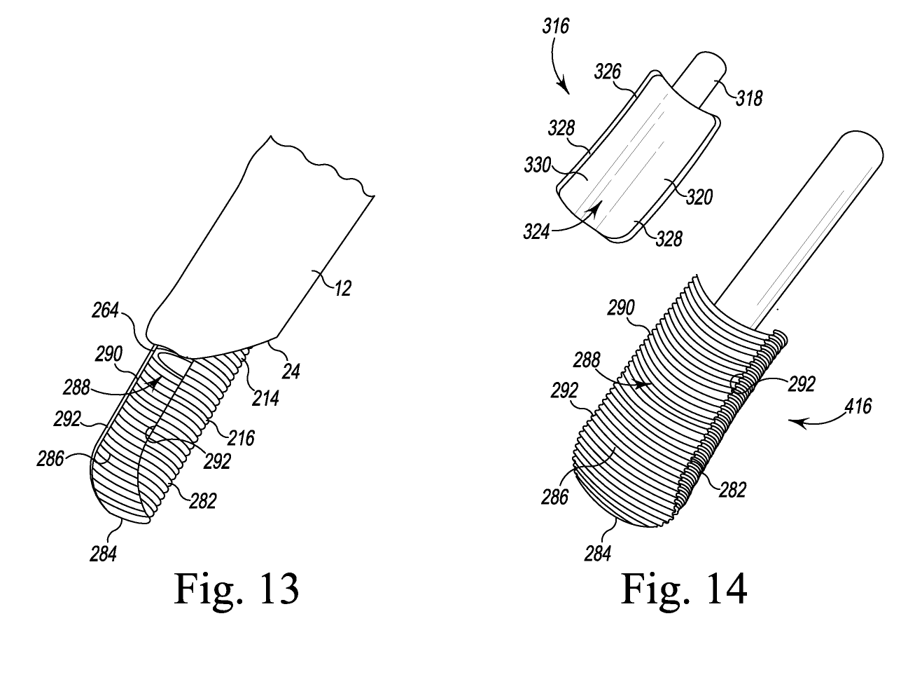

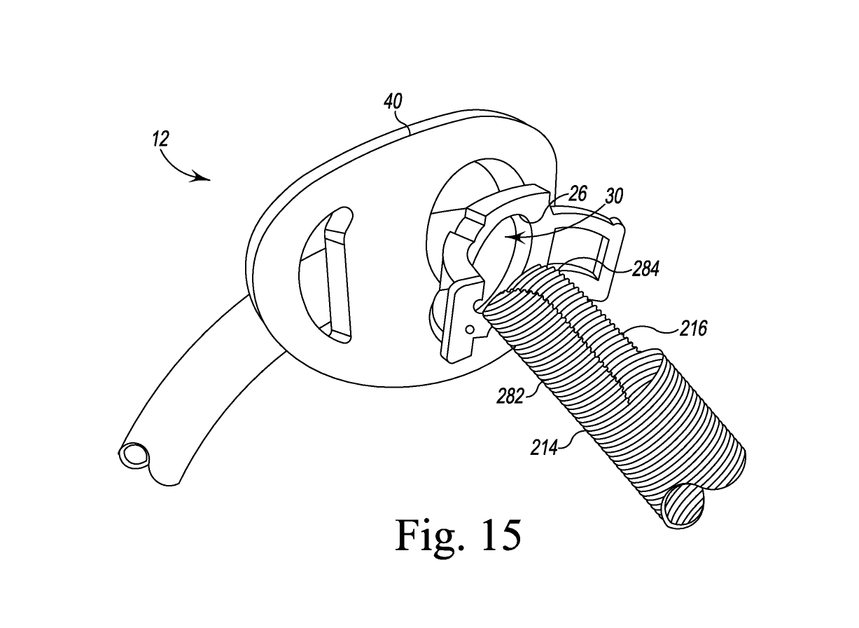

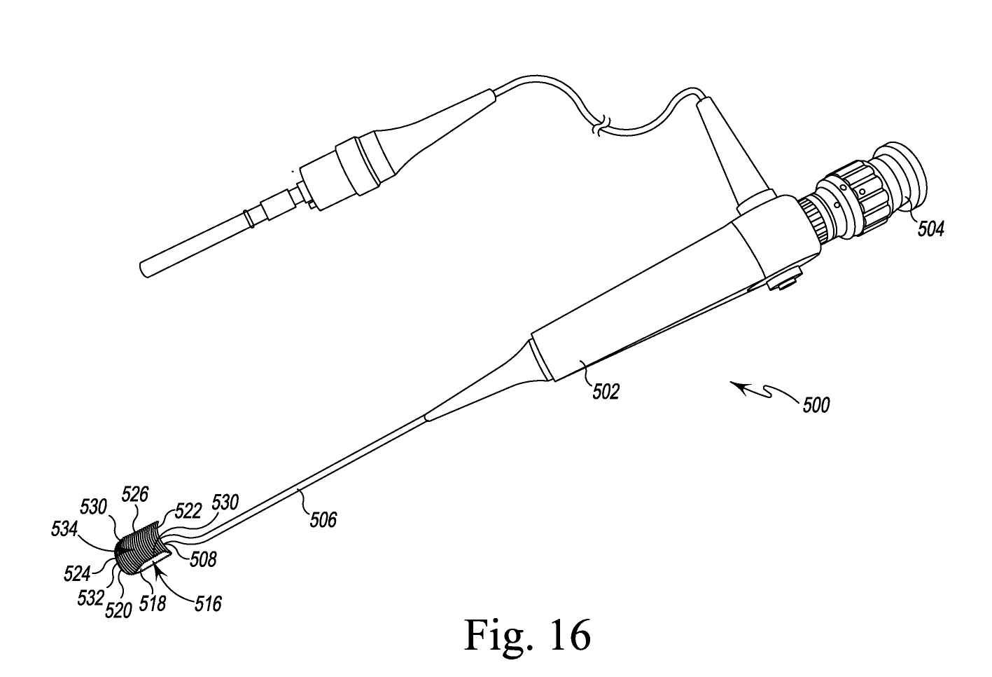

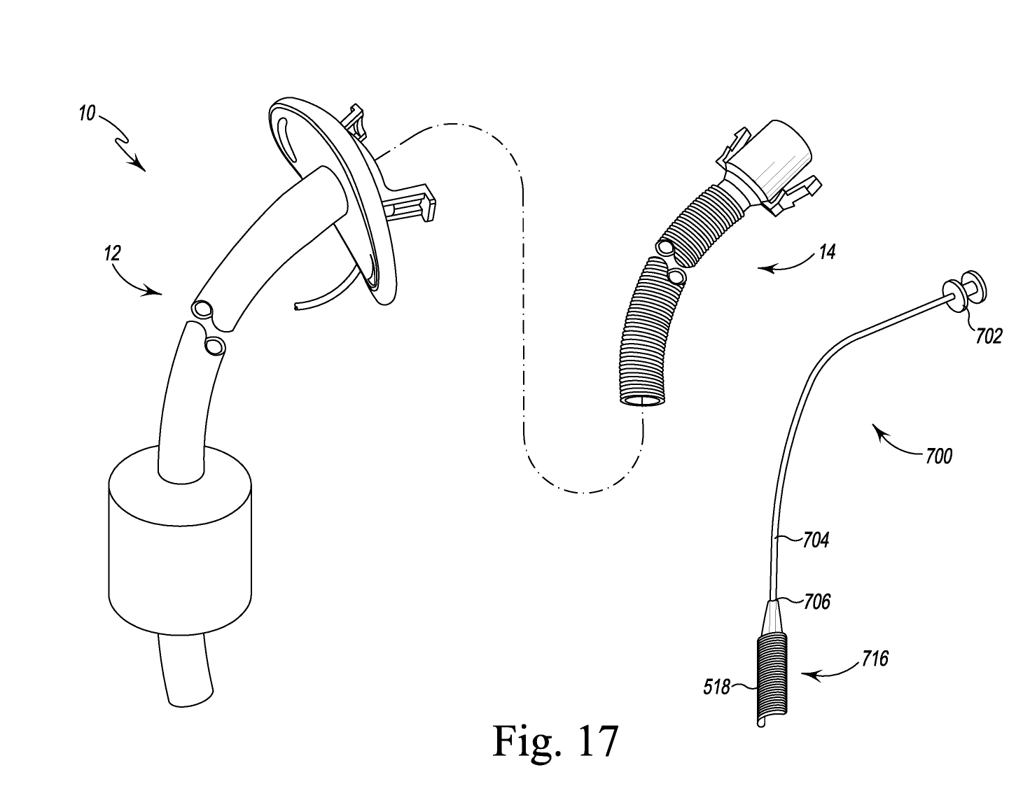

Referring now to FIG. 13, the system 10 may include a laryngoscope 500 and a tissue shield 516. The scope 500 includes an elongated handle 502 and a viewport 504 secured to one end of the handle 502. A cable 506 extends away from the opposite end of the handle 502 and has a probe tip 508 configured for insertion into a patient's trachea and/ or an endotracheal tube such as, for example, the endotracheal tube 12 described above. In the illustrative embodiment, the tissue shield 516 is attached to the probe tip 508. It should be appreciated that in other embodiments the tissue shield may be configured to be detached from the probe tip 508.

The tissue shield 516 includes a barrier plate 518 that extends from the probe tip 508 to a distal tip 520. In the illustrative embodiment, the barrier plate 518 includes a corrugated body 522 that extends to the distal tip 520. A corrugated body as an open end 524 at the distal tip 520, and an elongated slot 526 that extends proximally from the open end 524. The slot 526 is defined between a pair of edges 530 of the barrier plate 518, and the barrier plate 518 has a curved inner wall 532 that extends between the edges 530. The inner wall 532 defines a channel 534. The channel 534 and the elongated slot 526 are sized to receive a cutting tip of a percutaneous needle or other cutting tool.

Referring now to FIG. 14, the system 10 may include a stylette 700 sized to be positioned in an elongated tube 14 and/ or an endotracheal tube 12 and having sufficient length to extend outwardly from the distal opening of the endotracheal tube 12. The stylette 700 includes a grip 702 and a cable 704 that extends outwardly from the grip 702. A tissue shield 716 is attached at the distal end 706 of the cable 704 and is configured to extend outwardly from the distal opening of the endotracheal tube 12 when the stylette 700 is properly positioned. The tissue shield 716 includes a barrier plate 518, which has a channel 534 and an elongated slot 526 that are sized to receive a cutting tip of a percutaneous needle or other cutting tool. The cable 704 of the stylette 700 is sized so that blockage of the lumen 30 of the endotracheal tube 12 or passageway 76 of the elongated tube 14 is avoided.

It should be appreciated that, although the tissue shields shown and described above each include curved inner walls that define the channel that receives the cutting tip of a cutting tool, in other embodiments the inner wall may be substantially flat or take other shapes suitable for engaging a needle, catheter, or other surgical or medical tool to capture or redirect those tools away from the patient's soft tissue. It should also be appreciated that in other embodiments other biocompatible materials having sufficient strength and rigidity may be used to make the tissue shields.

While the disclosure has been illustrated and described in detail in the drawings and foregoing description, such an illustration and description is to be considered as exemplary and not restrictive in character, it being understood that only illustrative embodiments have been shown and described and that all changes and modifications that come within the spirit of the disclosure are desired to be protected.

There are a plurality of advantages of the present disclosure arising from the various features of the method, apparatus, and system described herein. It will be noted that alternative embodiments of the method, apparatus, and system of the present disclosure may not include all of the features described yet still benefit from at least some of the advantages of such features. Those of ordinary skill in the art may readily devise their own implementations of the method, apparatus, and system that incorporate one or more of the features of the present invention and fall within the spirit and scope of the present disclosure as defined by the appended claims.

1. A medical instrument system comprising:
an endotracheal tube including a distal end sized to be positioned in a patient's trachea, a proximal end configured to extend outwardly from one of the patient's mouth and the patient's nose, and a lumen extending between the distal end and the proximal end, and
a tissue shield sized to be advanced into the lumen of the endotracheal tube, the tissue shield including a barrier plate sized and shaped to extend outwardly the distal end of the endotracheal tube and block a medical tool from contacting a posterior wall of the patient's trachea.

2. The medical instrument system of claim 1, further comprising:
an elongated tube sized to be positioned in the lumen of the endotracheal tube,
wherein the tissue shield includes a cylindrical body sized to be received a passageway defined in the elongated tube, and the barrier plate is secured to, and extends distally away from, the cylindrical body.

3. The medical instrument system of claim 2, wherein:
the elongated tube includes a plurality of corrugations, and
the cylindrical body of the tissue shield includes a rib sized to be positioned in one of the corrugations to secure the shield to the elongated tube.

4. The medical instrument system of claim 1, further comprising:
a medical instrument including a cable sized to be advanced into the lumen of the endotracheal tube,
wherein the tissue shield is secured to a distal end of the cable of the medical instrument.

5. The medical instrument system of claim 4, wherein the medical instrument includes a laryngoscope.

6. The medical instrument system of claim 4, wherein the cable is a stylette.

7. The medical instrument system of claim 1, further comprising an elongated tube sized to be positioned in the lumen of the endotracheal tube, wherein the tissue shield is secured to a distal end of the elongated tube.

8. The medical instrument system of claim 7, wherein the elongated tube and the tissue shield are formed as a single monolithic component.

9. The medical instrument system of claim 7, wherein the tissue shield is configured to be detached from the elongated tube.

10. The medical instrument system of claim 1, wherein the barrier plate includes:
a curved wall that extends around a longitudinal axis from a first edge to a second edge, and
an opening is defined between the first edge and the second edge of the curved wall, the opening being sized cover a predetermined portion of a patient's soft tissue.

11. The medical instrument system of claim 10, wherein the curved wall is formed from a semi-flexible plastic.

12. The medical instrument system of claim 10, wherein the curved wall is formed from silicone.

13. The medical instrument system of claim 1, wherein the endotracheal tube includes an inflatable cuff positioned between the distal end and the proximal end.

14. A medical instrument system comprising:
an elongated tube sized to be positioned in an endotracheal tube, the elongated tube having a distal end, a proximal end, and an outer surface devoid of any openings between the distal end and the proximal end, and
a tissue shield secured to the distal end of the elongated tube, the shield including a barrier plate sized and shaped to block a cutting tool from puncturing a posterior wall of a patient's trachea.

15. The medical instrument system of claim 14, wherein the elongated tube and the tissue shield are formed as a single monolithic component.

16. The medical instrument system of claim 14, wherein the tissue shield is configured to be detached from the elongated tube.

17. The medical instrument system of claim 16, wherein the tissue shield includes a cylindrical body sized to be received a passageway defined in the elongated tube, and the barrier plate is secured to, and extends distally away from, the cylindrical body.

18. The medical instrument system of claim 17, wherein:
the elongated tube includes a plurality of corrugations, and
the cylindrical body of the tissue shield includes a rib sized to be positioned in one of the corrugations to secure the shield to the elongated tube.

19. A medical instrument system comprising:
a surgical instrument including a cable sized to be advanced into an endotracheal tube, and
a tissue shield secured to a distal end of the cable, the tissue shield including a barrier plate sized and shaped to prevent a cutting tool from puncturing a posterior wall of a patient's trachea.

20. The medical instrument system of claim 19, wherein the surgical instrument includes a laryngoscope.

21. The medical instrument system of claim 19, wherein the cable is a stylette.

22. A method of forming an incision in a patient's neck in advance of implanting a tracheal device, the method comprising:
sliding an endotracheal tube to move the distal end of the endotracheal tube proximally within the patient's trachea,
moving a tissue shield outward from the distal end of the endotracheal tube to position a barrier plate of the shield in the patient's trachea between an anterior wall of the patient's trachea and a posterior wall of the patient's trachea,
inserting a surgical tool through the anterior wall of the patient's trachea to form an incision, and
advancing the surgical tool into engagement with the barrier plate of the shield.

23. The method of claim 22, further comprising:
withdrawing an elongated tube from the endotracheal tube, and
attaching the tissue shield to a distal end of the elongated tube,
wherein moving the shield outward from the distal end of the endotracheal tube includes inserting the elongated tube with the tissue shield attached to its distal end into the endotracheal tube.

24. The method of claim 22, further comprising:
inserting the tissue shield attached to a distal end of a laryngoscope into the endotracheal tube, and
advancing the tissue shield and the distal end of the laryngoscope toward the distal end of the endotracheal tube.

25. The method of claim 22, further comprising:
inserting the tissue shield attached to a distal end of a stylette into the endotracheal tube, and
advancing the tissue shield and the distal end of the stylette toward the distal end of the endotracheal tube.
